(19) United States
(12) Patent Application Publication
(10) Pub. No.: US 2021/0402372 A1
(43) Pub. Date: Dec. 30, 2021

(54) WASHABLE FLUID ABSORBING COMPOSITE MATERIAL

(71) Applicants: Michael BERGMAN, Kityat Gat (IL); Yael BOOCK CARMONA, Herzlia (IL)

(72) Inventors: Michael BERGMAN, Kityat Gat (IL); Yael BOOCK CARMONA, Herzlia (IL)

(21) Appl. No.: 17/288,710

(22) PCT Filed: Oct. 27, 2019
(86) PCT No.: PCT/IB2019/059198

§ 371 Date: Apr. 26, 2021

Related U.S. Application Data
(60) Provisional application No. 62/751,579, filed on Oct. 27, 2018.

(51) Int. Cl. B01J 20/28; B01J 20/24; B01J 20/26; A61L 15/52; A61L 15/24; A61L 15/26; A47G 9/02; A47C 31/10

(52) U.S. Cl. CPC B01J 20/2805; B01J 20/28038; B01J 20/24; B01J 20/262; A61L 15/52; A61F 5/48; A61L 15/26; A47G 9/0246; A47G 9/0261; A47G 9/0253; A47C 31/105; A61L 15/24

(57) ABSTRACT

A washable multi-layer composite capable of fluid containment that may be used to protect a surface from liquid infiltration

### FIELD OF THE INVENTION

The present invention relates to multi-layer composite material, and in particular, to a washable composite material capable of fluid containment that may be used to protect a surface from liquid infiltration.

### BACKGROUND OF THE INVENTION

Urinary incontinence afflicts many individuals of all age groups. The severity and type of urinary incontinence is greatly variable among the population. One form of incontinence common in children is night time wetting or bedwetting.

The problem faced by those suffering from urinary incontinence and their caregivers is not only measured by the nuisance it entails in having to repeatedly clean bedding, clothing on a regular basis, in some instances multiple times in a single day. Many times urinary incontinence has a profound effect on the quality the of life both for caregivers and for those suffering. Moreover, this problem entails and associated with both social and psychological effects that afflict both caregivers and those suffering from urinary incontinence. Such quality of life, social, and psychological problems are sometimes more problematic and/or severe than the source of the problem. Such a problem greatly limits individuals, particularly kids, as urinary insentience is associated with shame, social ridicule, and reduced social interactions.

Presently available commercial products address the various levels of urinary incontinence. The commercial products are found in the form of pads, diapers and the like that intend to absorb urine with a moisture absorbing component.

Incontinence pads are generally provided in one of two forms either a relatively inexpensive and disposable pad, or, an expensive and reusable pad.

Such prior art multi-use pads typically include a moisture absorbing component combined with an overlying moisture barrier. The moisture barrier is intended to prevent or limit the movement of moisture through the moisture absorbing component and out of the pad. However, a common drawback occurs when attempting to wash such pads for reuse, where it becomes apparent that it is very difficult to fully clean the moisture absorbing component. Specifically, such pads cannot fully remove the urine's liquid portion and/or the urine's solids. Traces of urine constituents, liquid or solid, remain in the pad and are not efficiently removed. Such solids when remaining in the pads lead to the identifiable “smell” associated with urine. This limitation is primarily due to the pad's construction which, by design, restricts flow of water through the pad caused by the moisture barrier.

Laundering the pad therefore presents the reverse problem, how to effectively remove the fluid, and associated odor, from within the pad.

This problem persists as incontinence pads are designed to receive the fluid, but are unable to efficiently and effectively release the fluid so as to allow the pad to dry. It is believed that this problem is due because the pad cannot remove the fluid retained in the absorbing layer after washing. Generally the urine remains trapped and/or locked into the central layers. This prolongs the drying period and is particularly inefficient in removing the odors associated with urine.

Therefore although an advantage of such re-usable pads lies in their repeated use, however, the considerable time and inconvenience in laundering generally outweighs the ultimate costs savings.

Furthermore, when saturated, existing pads generally trap moisture directly against the skin of the user creating an uncomfortable and unhealthy environment prone to fungus and bacteria growth.

While some solutions have been proposed they do not provide a competent solution as the laundering problem persists as the pads are not easily washed. Furthermore once dry the washed fabric becomes deformed and does not retain and/or re-assume its original shape, forma and function. U.S. Pat. No. 7,361,803 to Miskie, provides a multi-layer moisture management fabric composite that is fit within a jacket wherein the layers forming the fabric are incompletely coupled and/or attached to one another. The fabric features fixed end and a free end. This configuration allows the individual layers to be loose and/or substantially unattached with one another. Such a configuration renders the fabric non-uniform and proves to be inefficient for the washing and drying as layers behave in an individual manner in each of the layers.

Furthermore, such prior art multi-layer fabric are inefficient as it requires a user to disassemble the fabric prior to washing and thereafter reassemble by a user following washing and drying. This process renders the prior art pad an inconvenient and/or inefficient solution. Furthermore, the proximity of the different layers that are placed one below the other results in the eventual migration of the liquid movement in both directions where liquid travels toward a user, especially once the fibers have become somewhat saturated. At that time the fabric's properties change in such a manner that they can no longer control the moisture flow away from the user leading to moisture seeping back toward the user.

### SUMMARY OF THE INVENTION

The background art does not provide a thorough solution for urinary incontinence and therefore does not facilitate alleviating the problem itself or the associated quality of life, psychological and social complications.

Embodiments of the present invention overcome the functional deficiencies of the background art and further provide improvement in the quality of life, psychological and social aspects for both those suffering from incontinence and their caregivers just the same.

The present invention provides a urinary incontinence solution provides an incontinence product that is readily washed and dried; provides absolute protection from fluid infiltration of the mattress and bedding; prevents side leakage; securely associated with and fits over a bed; has the look and feel of a printed bed sheet, which contributing to improvement in the quality of life, social and psychological factors; provides cost savings by virtue of ability to undergo many washing cycles while remaining functional.

The present invention overcomes the deficiencies of the background by providing a fluid absorbing washable multi-layer composite material that retains its characteristics both prior to and following a washing and drying cycle. Furthermore, the composite material of the present invention provides an easy to use and convenient material that may be readily securely to a surface, for example a mattress.

The composite material of the present invention provides a convenient material that may be readily secured to a surface, such as a mattress, while being used as a protective fluid absorbing and washable material.

In embodiments, the composite material may be provided in any form and in particular may be provided in the form of a bed sheet that can manage nocturnal urinary incontinence event and further allows re-use of the composite material while maintaining the composite material's properties over time, in particular, fluid containment and protective properties over time.

In embodiments, the composite material of the present invention is configured to keep both the user and the support surface on which the material is placed, dry.

In embodiments, the composite material is capable of maintaining its shape and form after washing. The composite material configuration of the present invention therefore provides a composite material that is competent for multiuse for individuals suffering from urinary in continence.

In embodiments the composite material may be associated with and/or placed on a plurality of optional surfaces to protect it from infiltration by a liquid such as water and/or urine. The protected surface may for example include but is not limited to mattresses, bed, sofa, couch, chairs, seat, high chairs, car seats, child safety car seats, child boosters, floor, table, or the like.

In embodiments the multi-layered composite material may be utilized to form a plurality of optional bedding textiles for example including but not limited to bed sheet, fitted bed sheet, pillowcase, pillow sham, duvet cover, blanket, bottom sheet, top sheet, cover, the like or any combination thereof.

In embodiments the multi-layered composite material may be utilized to form a plurality of optional materials for example including but not limited to: floor mat, carpeting, bathroom floor mat, the like or any combination thereof.

In embodiments the multi-layer composite material may include at last one upper surface may adorn a pattern and/or ornamental design. For example, the composite material may be provided in the form of a fitted bed sheet onto which an ornamental design and/or logo for example including but not limited to a superhero, a sports figure, team logo, or the like may be printed and/or embedded about the upper surface. For example a bedsheet for a child may include state of the art design and/or logos that are associated with favorite sports teams, favorite movies, characters, any combination thereof or the like.

Within the context of this application the term ‘GSM’ refers to a fabric weight measure referring to the fabric's weight in grams per square meter.

Within the context of this application the term denier or den or ‘D’ may be used interchangeably as is known in the art to refer to a unit of measurement indicative of the linear mass weight of textile fibers per 9000 meters of filament, where one denier is equal to about 0.11 mg/m of filament.

Within the context of this application the terms ‘tex’ and/or ‘dtex’ and/or ‘decitex’ may be used interchangeably as is known in the art to refer to a unit of measurement indicative of the linear mass weight of textile fibers per 1000 meters of filament for ‘tex’, and per 10000 for ‘dtex’ or ‘decitex’.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A-B are illustrative schematic diagram of an exemplary fluid protective composite materials according to the present invention;

FIG. 1C shows an illustrative schematic diagram showing a close up view of an end of the composite material according to the present invention;

FIG. 2A-C are illustrative schematic diagrams of the composite multi-layered fabric showing the layered arrangement according to embodiments of the present invention.

### DESCRIPTION OF THE PREFERRED EMBODIMENTS

The present invention provides a multi-layer a washable composite material that forms a system capable of fluid containment and used as a protective material capable of protecting a surface onto which the composite is placed.

In a preferred embodiment, the present invention provides a bedding composite material forming a system that is useful for covering a surface such as a bed, for example provided in the form of a sleeping sheet. Such a system is useful in helping individuals and/or caregivers of individuals that suffer from urinary incontinence, and in particular night wetting known as nocturnal urinary incontinence. Accordingly, the multi-layered composite material provided by embodiments of the present invention provides for surface protection of a bed and/or mattress and/or sofa from urine infiltration.

Furthermore the multi-layered composite material of the present invention provides a re-usable and/or washable bed sheet and/or fabric that maintains its form, structure and fluid containment capabilities over time.

The principles of the present invention may be better understood with reference to the drawings and the accompanying description. The following figure reference labels are used throughout the description to refer to similarly functioning components are used throughout the specification hereinbelow.

10 protected surface;

100 composite fabric;

101 fitting flaps/skirt;

102 coupling seam;

104 medial portion/internal volume;

106 handle;

106a,b first and second handle portions;

108 internal volume entrance/opening;

110 upper portion;

112 first layer; user

114 second layer;

120 lower portion;

122 third layer;

124 fourth layer;

126 fifth layer;

128 pooling side walls;

130 inner volume end seal;

132 inner volume end seal stitch;

134 seam sealant;

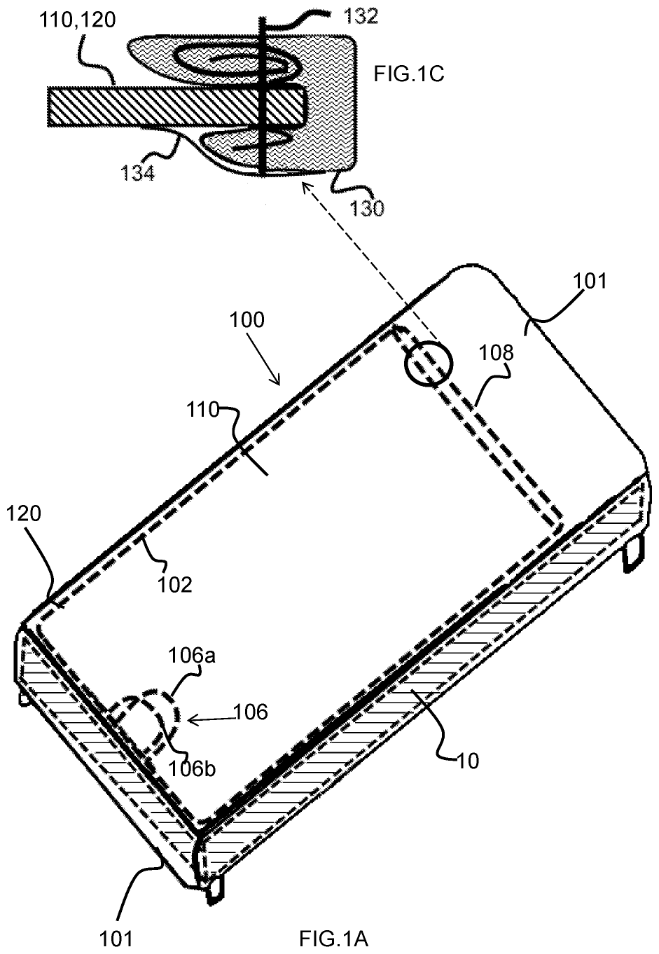

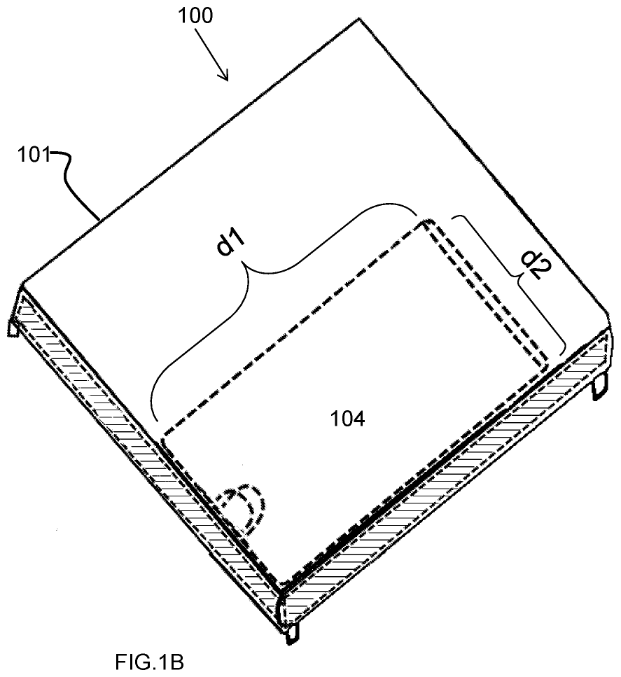

FIG. 1A shows an illustrative diagram of use of the multi-layer composite material 100 that is fit over a surface 10, shown in the form of a bed and/or mattress. Composite 100 is provided so as to protect surface 10 from fluid infiltration, in particularly urine.

Accordingly a preferred embodiment of the present invention provides a multi-layered composite material 100 that may be provided in the form of a fitted bed sheet that properly and tightly fits over a mattress 10 and is capable absorbing liquids, such as urine, to provide protection for the underlying mattress 10 from urine infiltration.

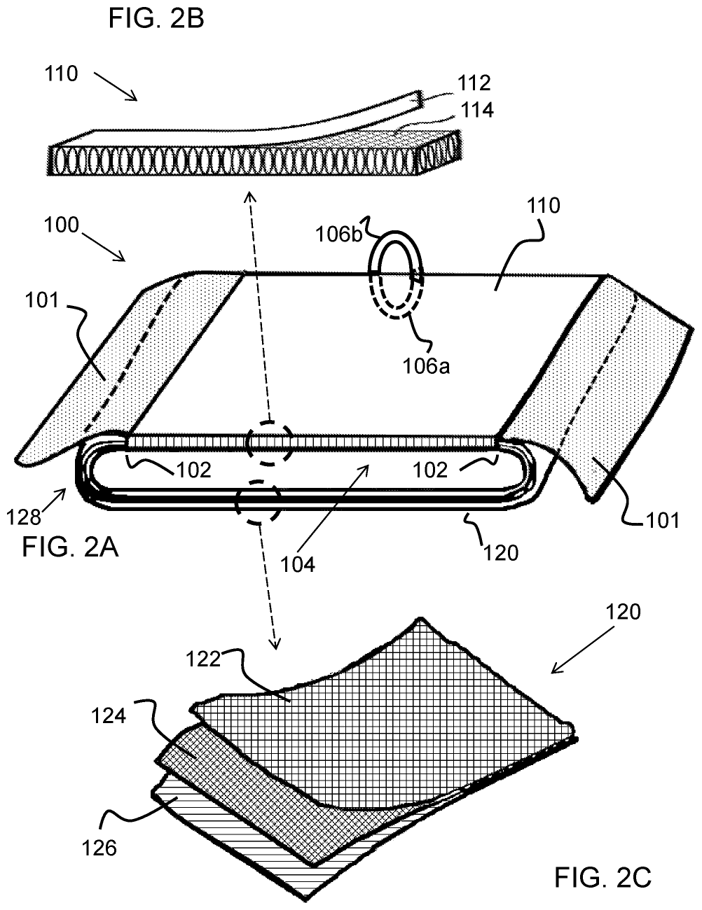

In some embodiments the proper and tight fit of composite 100 about surface 10 is ensured with the use of a high modulus elastic cord (not shown). Most preferably the high modulus elastic cord or the like securing means allows system 100 to bed secured over support surface 10, for example a mattress, so as to ensure performance of composite 100. The elastic cord is optionally and preferably provided along the fitted skirt portion 101, shown in FIG. 2A, utilized to ensure that composite 100 fits with surfaces of varying heights. In some embodiments the elastic cord is provided with a stopper (not shown) to properly adjust the length of the elastic cord.

FIG. 1B shows a similar view as that shown in FIG. 1A however providing a schematic illustration of the composite material 100 that is fitted over one side of a wider mattress, for example a double, queen and/or king sized mattress. Composite 100 may be provided in optional sizes and dimensions and capable of fitting over the entire width of the mattress size, as shown in FIG. 1A. In some embodiments composite 100 may be configured to protect a select portion and/or side of a mattress for example as shown in FIG. 1B where only one side of the fitted bed sheet is provided with composite 100. Optionally when a select portion of surface 10 is selected to be protected with composite material 100 the corresponding protected area of surface 10 and composite material 100 may be fit with hook and look, VELCO®, or the like coupling means to selectively and securely couple composite material 100 over the selected portion of surface 10. For example as shown in FIG. 1B where only the right side of the mattress 10 is protected with composite 100, accordingly that area may feature corresponding hook and loop structure to increase coupling of composite 100 over the right side of the mattress.

In embodiments the composite 100 may be formed of different sizes and/or shapes. For illustrative purposes reference is made with respect to FIG. 1B to depict sizing option of composite 100. Composite 100 preferably includes a fitting area/zone defined by skirt 101 and a protected area/zone defined by the area formed by inner volume 104.

Preferably the area to be protected is defined by the area underlying inner volume 104 defined by a length d1 and a width d2 as schematically shown in FIG. 1B. Accordingly the protected area/zone may be defined by controlling the relative dimensions d1 and d2 to form any shape.

The area/zone to be protected corresponding to inner volume 104 may have any shape or form for example including but not limited to rectangular, quadrilateral, polygonal, elliptic, ovoid, circular, or the like shape.

In embodiments, the optimization of protected area relative to fitting area may be controlled by adjusting the relative proportions and/or ratio of area utilized to form inner volume 104 and fitted skirt area 101. For example, composite 100 may be configured so as to provide a protective zone defined by the area of inner volume 104 so as to cover the entire surface of surface 10, optionally covering the entire area of a surface/mattress 10 or a select portion of the area defined by surface 10.

In some embodiments the ratio of protected zone underlying inner volume 104 relative to fitted skirt area may be determined by the shape and/or function of the support surface 10 to be protected.

In embodiments, composite 100 is formed from an upper portion 110 that is in contact with the user, referred to as the user side, and a lower portion 120, details of which are best seen in FIG. 2A-C, that is in contact with the protected surface 10, shown in the form of mattress, and features an internal volume 104, seen in FIG. 2A-C. In optional but preferred embodiments composite 100 comprises a fitting flaps 101 and/or skirt that provides for snuggly securing and/or coupling composite 100 over the support surface 10, such that it acts as a uniform sheet covering a surface such as a bed and/or mattress.

As shown here, a fitting flaps 101 and/or skirt is provided in the form of flaps including a strong, high modulus elastic to ensure that composite 100 is snugly fit and secured onto mattress 10. In optional embodiments the size of skirt portion 101 may be controlled so as to allow composite 100 to fit over variously sized surfaces 10 for example twin, single, double, queen, king sized mattress, for example as shown in FIG. 1A-B. FIG. 1A shows use of composite 100 over a single and/or twin sized mattress 10 while FIG. 1B shows use of a composite provided to fit over one side of a double, queen, or king sized mattress and/or bed. The embodiment depicted in FIG. 1A utilizes a skirt 101 that is smaller than the skirt utilized in the embodiment depicted in FIG. 1B. Accordingly, by way of varying the dimensions, size and/or shape of skirt 101, composite 100 may be used on optional surfaces 10.

As shown, a preferred embodiment of composite 100, is provided in the form of an incontinence bed sheet that is capable of absorbing urine expelled during a nocturnal incontinence incident. Composite 100 is snugly fit over surface 10 shown in the form of a mattress providing protection for the mattress.

Composite 100 features an internal volume 104, discussed in greater detail with respect to FIG. 2A-C, that provides for facilitating reuse of composite 100 by enabling washing of composite 100. Specifically, amongst other features discussed later, internal volume 104 provides for turning composite 100 inside out prior to washing.

Accordingly, composite 100 features an opening 108 providing access to internal volume 104, wherein internal volume further features a looped handle 106 to facilitate turning composite 100 inside-out prior to washing. Preferably handle 106 comprises a first portion 106a and a second portion 106b that are utilized to switch between wash mode (inside out) and use mode. Preferably first portion 106a is used to turn composite 100 inside-out prior to a washing cycle while second portion 106b is used after drying cycle to reverse the configuration re-establishing internal volume 104.

Preferably opening 108 is provided by forming an envelope between upper portion 110 and lower portion 120.

In embodiments the edge forming opening 108 is preferably sealed as is shown in FIG. 1C, so as to prevent leakage from the edge forming opening 108. Fluid resistant end seal 130 is provided to ensure that fluids to not leak and/or escape from the end of the respective layers 110,120. Accordingly, the end edge of portions 110, 120 that forms opening 108 are sealed with a fluid resistant material 130 forming an inner volume end seal. Preferably, the fluid resistant end seal material 130 is preferably sewn onto portions 110, 120 utilizing a stitch 132. Preferably material 130 is folded over itself at the end edge of layers portion 110, 120 and thereafter stitched with stitch 132. Most preferably following stitching a seam sealant 134 is applied over stitch 132 so as to ensure that leaking does not take place through stitch 132. More preferably seam sealant 134 is provide over at least a lower surface material 130 so as to ensure that fluids do not escape through stitch 132. Optionally seam sealant 134 may be applied to both upper and lower surface of fluid resistant material 130 featuring stitching 132.

Seam sealant 134 may optionally be provided in the form of a seam seal tape as is known in the art. Optionally seam sealant may be provided in the form of a multilayered adhesive film, a heat sensitive sealant, a chemical agent, the like or any combination thereof.

In some embodiment end seal 130 may be provided from materials forming layers 124 and 126 that are folded over the edge of lower and/or upper portions 110,120, prior to applying stitch 132 and seam sealant 134.

In some embodiments opening 108 may be further fit with at least one or more closure means for example including but not limited to a zipper and/or buttons and/or hook and loop and/or overlapping flaps, waterproof flap coupled to 120 folded over 122 and coupled to 114 with hook and loop, waterproof zipper, airproof zipper, the like or any combination thereof for controllably opening and closing opening 108.

In some embodiments opening 108 may be provided along the length of one of the edges of composite 100 or along a portion of the length of one of the edges of composite 100. Preferably the length of opening 108 is determined based on the length required to facilitate turning composite 100 inside-out, with handle 106, to expose internal volume 104.

In preferred embodiment fitting flap 101 is integrated with composite 100 by way of sewing, over a seam 102. Optionally, fitting flap/skirt portion 101 may be integrated by sewing it between upper portion 110 and lower portion 120 so as to properly secure all layers together. Optionally skirt 101 may be fit with composite 100 based on the surface onto which it is to be used.

In optional embodiment, fitting skirt 101 may take any form that facilitates adhering composite 100 onto optional support surfaces. For example, fitting skirt 101 may be provided in the form of a chair and/or sofa to facilitate placing and/or fitting composite 100 over the relevant support surface 10.

In embodiments composite 100 may be utilized to protect a variety of surfaces 10 for example including but not limited to mattresses, bed, sofa, couch, chairs, high chairs, car seats, child safety car seats, child boosters, table, floor mat, carpeting, bathroom floor mat or the like.

In embodiments the multi-layered composite fabric may be utilized to form a plurality of optional protective and/or bedding textiles for example including but not limited to bed sheet, fitted bed sheet, pillowcase, pillow sham, duvet cover, blanket, bottom sheet, top sheet, cover, lawn furniture cover, the like or any combination thereof.

FIG. 2A-C shows a close up cross sectional view of the composite 100 according to the present invention revealing the multiple layers 112,114, 122, 124, 126 forming composite 100. The fabric layers are coupled over a coupling seam 102 to form three distinct portions 104, 110, 120. FIG. 2A shows a cross sectional view of composite 100 according to the present invention. FIG. 2B shows a close up view of the layers forming upper portion 110, and FIG. 2C shows a close up view of the layers forming lower portion 120.

The three portions 104, 110, 120 include an upper portion 110 that is in direct contact with the user, a lower portion 120 this is in direct contact with the support surface 10, such as a mattress, bed, sofa, or the like support surface and a medial and/or intermediate portion 104 defining an internal volume separating the upper portion 110 and lower portion 120.

Now referring to FIG. 2B showing, upper portion 110 that comprises two layers, a first layer 112 and a second layer 114. Layers 112 and 114 that are fused together as shown by seam 102. Upper portion 110 is the user side surface defining the surface on which the user interfaces, for example sleeps or sits. Specifically the user comes in direct contact with first layer 112. Preferably first layer 112 is a comfortable fabric having a nice touch and feel. Optionally first layer is configured such that it may be provided with a design and/or prints so as to render the composite 100 visually appealing to the respective audience and/or user. For example, a print of a comics character, superhero and/or princess may be provided on surface 112 so as to appeal to a young audience. Similarly, a floral pattern may be utilized on surface 112 so as to render composite 100 appealing for adult and/or elderly users.

First layer 112 is preferably a hydrophobic layer made of hydrophobic materials promoting quick drying and downward drainage (gravitational) flow of fluids into the underlying layers. Most preferably porosity of the hydrophobic layer that allows quick drainage of the urine and does not form a surface layer that prevents urine from draining downward. First layer 112 is provided from at least one or more of polyester, nylon, nylon-6, nylon-66, polypropylene, microfiber multi filament fibers, the like hydrophobic material or any combination thereof. In embodiments, first layer may be made from 100% of the hydrophobic material.

Layer 112 may be hydrophilic from microfiber that is very thin of at most 80 GSM, so that it has quickly absorbs and drains it is preferably the lowest possible GSM.

In some embodiments layer 112 may be a mixture hydrophilic and hydrophobic fibers in a ratio of 20:80, 10:90, 30:70, 40:60; 50:50

In embodiments layer 112 is provided with the proper ratio of hydrophilic and hydrophobic fibers so as to be render it both comfortable to touch and simultaneously functional so as to allow quick drainage of liquids, for example urine, into the underlying lower layers so as to maintain layer 112 as dry as possible.

In embodiments, first layer 112 may be woven, weave, weft knit or a warp knit, or any combination thereof.

In embodiments first layer 112 may have a weight ‘GSM’ from about 40 GSM up to about 140 GSM.

In embodiments second layer 114 is a three dimensional space layer (‘3D’ spacer) that functions to allow passage of liquid, in particular urine, into internal volume 104 and lower portion 120 while preventing the liquid from migrating upwards back toward the user. Second layer 114 is a 3D warp knit or woven hydrophobic layer and made from at least one or more of polyester, nylon, nylon-6, nylon-66, the like hydrophobic thread or any combination thereof. Second layer 114 is configured to provide a rebound force so that it won't collapse or pump liquids toward 112. Accordingly when a user sits and/or moves it will not result in fluid moving up through second layer 114 to first layer 112. In embodiments second layer 114 is provided so as to withstand a vertical pressure and/or force so as to prevent contact between layer 112 with lower portion 120 and in particular that of the absorbing layer 122.

In embodiments, first and second layer may be sewn together along their edge along a seam 102 where all layers are sewn together as will be described in greater detail below.

As shown in FIG. 2C lower portion 120 is provided in the form of a three layer laminated composite including third layer 122, fourth layer 124 and fifth layer 126. Lower portion 120 provides for absorbing liquids flowing into internal volume 104 toward third layer 122 so as to protect support surface 10, for example a mattress, from liquid infiltration. Accordingly, lower portion 120 provides for retaining the liquid within the composite 100.

Third layer 122 is preferably provided in the form hydrophilic material that readily absorbs liquids. Third layer 122 may be configured to absorb a liquid volume of up to about 2500 cc. Third layer 122 may be provided in the from at least one or more materials, fibers and/or textiles for example including but not limited to cotton, silk, linen, flax, viscose, rayon, bamboo, soya, corn, cellulose, Modal, Tensel, Layosel, micro-polyester, micro-nylon, micro-nylon 66, the like absorbent textiles, fibers or any combination thereof.

In embodiments, third layer 122 may be provided in various optional forms of terrycloth, for example including but not limited to single side loop terry structure, one sided terry weft, warp knit, a weft knit terry, a woven terrycloth, a non-woven terry the like or any combination thereof.

In embodiments, third layer 122 provided from terry cloth such that the layer's surface area is maximized contributing both to improved washing and drying, saving in the number of detergents, and washing cycles needed to effectively clean layer 122.

In embodiments third layer 122 may be provided as a layer that is provided from various threads, as described above, that are interlaced by any optional methods for example including but not limited to woven, knit, braided, plaiting, the like, one sided terry weft, warp knit, or any combination thereof as is known in the art. In some embodiments the material forming third layer 122 may be non-woven.

In some embodiments, third layer 122 may be provided from synthetic fibers, any such synthetic fibers are provided in the form of a synthetic micro-fiber having a denier of less than 1 (‘den’<1). The filaments forming third layer 122 may be provided from a fiber with a SLIT cross section.

In embodiments third layer 122 is preferably configured to exhibit hydrophilic properties wherein the layer comprises an absorbing capacity of about 2.5 liter per square meter of fabric.

In embodiments due to internal volume 104 composite 100 may be turned inside-out so as to expose third layer 122 during washing cycle. Accordingly, when turned inside out third layer 122 is outwardly facing in order to facilitate washing and exposing the absorbing layer, specifically so as to allow any absorbed liquids and urine solid, for example urine, to be exposed to cleaning detergents facilitating cleaning and further facilitating the drying process thereafter.

In embodiments fourth layer 124 is provided as a liquid resistant membrane. Accordingly fourth layer 124 is provided to prevent urine smell from penetrating into the mattress. Accordingly, further layer is provided as a fluid barrier to prevent fluid, in particular urine, from penetrating into the support surface 10 (not shown) for example a mattress, sofa, or the like support surface.

Preferably fourth layer 124 is provided form at least one or more optional textile materials for example including but not limited to polyurethane (‘PU’), poly vinyl chloride (‘PVC’), ThermoPlastic Polyurethane (‘TPU’), PolyTetraFluoroEthylene (‘PTFE’), the like or any combination thereof.

In embodiments fourth layer 124 is provided so as to resist and/or withstand hydrostatic pressure of at least 4000 mm water pressure according to the AATCC-127 water resistance test protocol. Optionally further layer is further provides for may washing cycles at variable temperatures from 40 and up to 90 degrees Celsius.

In embodiments fourth layer 124 is provided with a GSM of about 40. In some embodiment fourth layer may be provided with a GSM of up to about 100 GSM.

Fifth layer 126 is provided as a protective layer for the fourth layer 124 that is disposed directly above it. Fifth layer 126 provides for protecting the fourth layer 124 from abrasion and/or mechanical damage and/or wear and tear that may be expected during cleaning in particular during laundering washing and drying. Accordingly, the fifth layer 126 ensures that the fourth layer 124 remain viable for an extended period for example so as to allow fourth layer to remain viable with a performance standard according to at least the AATCC-127 test protocol as previously described.

Fifth layer 126 preferably forms the lowest surface of the composite 100 and is directly in contact with the receiving support surface 10, for example including but not limited to mattress, sofa or the like.

In embodiments layer 126 may be provided from a hydrophobic non-abrasive fiber that is warp knit, woven, weft knit, woven and one sided brushed polyester, or the like.

Preferably fifth layer 126 is further provided to prevent slippage and/or movement of the composite 100, from the area intended to be protected from example as shown in FIG. 1B. In some embodiments, such as that depicted in FIG. 1B, composite 100 may be further fit with a hook and loop structure, along the perimeter of fifth layer 126 so as to further secure composite 100 to intended area of protection of surface 10.

In embodiments layers 126 may be provide with a GSM of about 90. Optionally fifth layer 126 may be provided with a GSM of up to about 200 GSM.

Lower portion 120 comprising third layer 122, fourth layer 124 and fifth layer 126 that are preferably laminated together to form a unitary surface forming lower portion 120, for example as shown.

Lower portion 120 is coupled to upper portion 110 to form composite 100 over a seam 102. As described in FIG. 1A-B a fitting skirt 101 may be sandwiched between portion 120 and 110 prior to sewing so as to form a coherent uniform material such a bed sheet, for example as shown in FIG. 1A-B.

In embodiments, coupling of portion 110 with lower portion 120 with seam 102 forms the medial portion 104 defining an internal volume, that both provides means for turning composite 100 inside-out medial portion 104 further acts as a spacer between upper portion 110 and lower portion 120. Medial portion 104 provides an internal volume that allows fluids conveyed through portion 110 (from user) to pool therein as the fluid is subsequently absorbed by lower portion 120. Accordingly volume 104 provides an intermediate pooling area to allow composite fiber 100 to receive a liquid volume in a short amount of time, flooding, while allowing the liquid to absorb within lower portion over a longer period of time, allowing the composite 100 to dry in a short amount of time.

In embodiments internal volume 104 may be provided with a volume and/or liquid capacity of up to about 4000 cc.

Most preferably forming internal volume 104 is facilitated by providing upper portion 110 with an area that is slightly smaller than the area of lower portion 120, forming pooling side walls 128 as shown in FIG. 2A.

In some embodiments side walls 128 have a length of up to about 1.5 cm.

In embodiments, coupling seam 102 provides for coupling upper portion 110 with lower portion 120 in a manner so as to form internal volume 104 and side walls 128. Coupling seam 102 provides for coupling corresponding edges of upper portion 110 and lower portion 120.

In embodiments, the composite material 100 of the present invention can provide a sheet having an absorbing capacity of about 2.5 L per meter, 4 Liters for a bed size of 90 cm by 200 cm which should be sufficient for a single night. This capability is attributed to the layered configuration of composite 100 and the pooling side walls 128 formed around volume 104.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

It should be noted that where reference numerals appear in the claims, such numerals are included solely for the purpose of improving the intelligibility of the claims and are no way limiting on the scope of the claims.

Having described a specific preferred embodiment of the invention with reference to the accompanying drawings, it will be appreciated that the present invention is not limited to that precise embodiment and that various changes and modifications can be effected therein by one of ordinary skill in the art without departing from the scope or spirit of the invention defined by the appended claims.

Further modifications of the invention will also occur to persons skilled in the art and all such are deemed to fall within the spirit and scope of the invention as defined by the appended claims.

While the invention has been described with respect to a limited number of embodiments, it will be appreciated that many variations, modifications and other applications of the invention may be made.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

While the invention has been described with respect to a limited number of embodiments, it will be appreciated that many variations, modifications and other applications of the invention may be made.

1)-26) (canceled)

27) A multi-layered liquid absorbing composite comprising: an upper portion, a medial portion and a lower portion:
a) the upper portion forming the body side surface of the fabric that provides a fluid permeable channeling portion capable of conveying a liquid from a user and allowing the liquid to flow toward the medial portion; said upper portion comprising a first layer and a second layer wherein said second layer is a non-wicking hydrophobic layer;
b) the medial portion forming an internal volume separating the upper portion form the lower portion; the medial portion featuring an opening and handle disposed internal to said volume;
c) the lower portion forming the support side surface of the fabric and providing for fluid retainment configured to retain fluid within the lower portion; wherein said lower portion is a multilayer composite comprising a liquid resistant membrane layer .
d) the composite characterized in that the upper portion and lower portion are coupled to one another along at least three corresponding outer edges forming an envelope that defines said internal volume and said opening, and further characterized in that the area of said upper portion is smaller than the area of said lower portion forming a pooling side walls.

28) The composite of claim 27 further comprising a fitting skirt.

29) The composite of claim 28 wherein said fitting skirt is disposed and sewn between said upper portion and lower portion along said seam.

30) The composite of claim 28 wherein said fitting skirt is a bedding fitted sheet comprising an elastic.

31) The composite of claim 28 wherein said fitting skirt further comprises a high modulus elastic cord and cord limiting stopper.

32) The composite of claim 27 wherein said internal volume is at least 4000 cc.

33) The composite of claim 27 provided in the form of bed cover or linen.

34) The composite of claim 27 provided in the form selected from duvet cover, top sheet, fitted sheet, bottom sheet, pillowcase, and pillow sham.

35) The composite material of claim 27 wherein said first layer is a hydrophobic layer provided from one of: hydrophobic materials, hydrophilic materials or a mixture of hydrophilic and hydrophobic fibers.

36) The composite material of claim 35 wherein said hydrophobic materials are selected from the group comprising one or more of: polyester, nylon, nylon-6, nylon-66, polypropylene, microfiber multi filament fibers, and any combination thereof.

37) The composite material of claim 27 wherein said first layer is provided with a weight from 80 GSM up to 120 GSM.

38) The composite material of claim 27 wherein said second layer is a three dimensional spacer layer (‘3D spacer’).

39) The composite material of claim 38 wherein said second layer is warp knit or a woven hydrophobic layer provided from fibers selected from one or more of: polyester, nylon, nylon-6, nylon-66.

40) The composite material of claim 38 wherein said second layer has a vertical pressure and/or force resistance of 80 gr/cm\hat{ }2 (eighty grams per centimeters squared).

41) The composite of claim 27 wherein said lower portion is a three layer laminate comprising a third layer, a fourth layer provided in the form of said membrane and a fifth layer.

42) The composite of claim 41 wherein said third layer is provided from hydrophilic materials capable of absorbing a liquid volume of up to 2500 cc per square meter.

43) The composite of claim 42 wherein said third layer is provided from materials selected from the group consisting of cotton, silk, linen, flax, viscose, rayon, bamboo, soya, corn, cellulose, Modal, Tensel, Layosel, micro-polyester, micro-nylon, micro-nylon 66, the like absorbent textiles, fibers, and any combination thereof.

44) The composite of claim 41 wherein said third layer is provided from terrycloth having a structure selected from the group consisting of single side loop terry structure, one sided terry, warp knit terry, a weft knit terry, a woven terrycloth, a non-woven, or any combination thereof.

45) The composite of claim 27 wherein said membrane is provided from polyurethane (‘PU’).

46) The composite of claim 27 wherein said membrane is provided from at least one or more materials selected from the group consisting of: polyurethane (‘PU’), poly vinyl chloride (‘PVC’), ThermoPlastic Polyurethane (‘TPU’), PolyTetraFluoroEthylene (‘PTFE’), or any combination thereof.

47) The composite of claim 27 wherein said membrane is capable of resisting a hydrostatic pressure of at least 4000 mm of water according to AATCC-127 water resistance protocol.

48) The composite of claim 41 wherein said fifth layer is a membrane protector layer provided for protecting said membrane during use, washing and drying cycles; wherein said fifth layer is provided from a hydrophobic non-abrasive fiber that may be formed by way of at least one of: warp knit, woven, weft knit, woven and one sided brushed polyester, or any combination thereof.

49) The composite of claim 41 wherein said fifth layer features an anti-slip surface along the undersurface that is in contact with the support surface.

50) The composite material of claim 27 wherein said opening features at least one or more closure means selected from the group consisting of a zipper, buttons, hook and loop, overlapping flaps, and any combination.

51) The composite material of claim 27 wherein an end of said opening is sealed with a fluid resistant material that is folded over an end edge of at last one of upper portion or lower portion that form said opening.

52) The composite material of claim 27 wherein said fluid resistant is coupled with an end edge of said upper portion or said lower portion with a stitch that is further sealed with a sealant along at least one of an upper and/or lower stitch surface.
